(19) United States
(12) Patent Application Publication
(10) Pub. No.: US 2003/0008334 A1
(43) Pub. Date: Jan. 9, 2003

(54) Protein nectin-3

(76) Inventors: Yoshimi Takai, Hyogo (JP); Hiroyuki Nakanishi, Hyogo (JP); Keiko Sato, Hyogo (JP); Kenichi Takahashi, Hyogo (JP)

Correspondence Address: WENDEROTH, LIND & PONACK, L.L.P., 2033 K STREET N. W., SUITE 800, WASHINGTON, DC, 20006-1021, US

(21) Appl. No.: 09/959,845

(22) PCT Filed: Mar. 9, 2001
(86) PCT No.: PCT/JP01/01871

(30) Foreign Application Priority Data
Mar. 9, 2000 (JP) 2000-065595

(51) Int. Cl. G01N 33/574; C07K 14/435

(52) U.S. Cl. 435/7.23; 530/350

(57) ABSTRACT

This application provides a novel protein, nectin-3 having an amino acid sequence of any of SEQ ID NO: 2, 4 or 6, which belongs to a nectin protein family participating in cell-cell adhesion. It also provides a polynucleotide that codes for the nectin-3 and has a base sequence of any of SEQ ID NO: 1, 3 or 5; a recombinant vector having the polynucleotide; and an antibody against the protein nectin-3. The protein nectin-3 provides important information for clarifying all aspects of the molecular mechanism in cell-cell binding systems, and, in addition, it leads to the possibility of clarifying the mechanism of, for example, humectation and metastasis of carcinoma, and is expected to be applicable to diagnosis of carcinoma for its malignancy and to a method for treating cases with carcinoma and also to development of medicines for carcinoma.

### TECHNICAL FIELD

The invention of this application relates to a novel protein nectin-3 that participates in cadherin-based cell-cell adherens junctions, and to gene-engineering materials for obtaining and utilizing the protein.

### BACKGROUND ART

Cell-cell adhesion systems to be formed by transmembrane proteins, such as adhesion molecules, receptors and channels play an important role in various cell-level phenomena such as cell-cell adhesion, cell movement and cell morphology determination in animal individuals. Above all, cell-cell adherens junctions (AJs) bear a role indispensable for histocompatibility. Evidence is increasing to say that AJs further participate in controlling cell propagation and morphologic tissue formation, in addition to their mechanistic role as above, It has become clarified that many F-actin-binding proteins play a role as a linker to link actin cytoskeletons to adhesion molecules. However, the molecule-level understanding of AJs is insufficient, and it is not clear as to which molecules may bind actin cytoskeletons to cell membranes.

To clarify this, the inventors of this application have isolated some novel F-actin-binding proteins from rats' brains, analyzed the structure of the protein especially specific to neurocytes and abundantly existing in synapses, and named it “neurabin”, for which the applicant already filed a patent application (Japanese Patent Application No. 276784/1998). Further, the inventors of this application have individually isolated 1-afadin, a novel F-actin-binding protein, and ponsin, a protein that binds with 1-afadin, for both of which the applicant filed patent applications (for 1-afadin, Japanese Patent Application No. 89572/1999; for ponsin, Japanese Patent Application No. 174687/1999). Still further, the inventors of this application have identified novel proteins, nectin-1α, 1β and nectin-2α, 2δ that function for cadherin-based AJ formation along with afadin (J. Cell Biol., 145:539-549, 1999). They have found that these nectin-1, -2 are Ca²⁺-independent immunoglobulin-like adhesion molecules and act on AJ formation in one system along with afadin and ponsin (Genes Cells 4:573-581, 1999; J. Biol. Chem., 275:613-618, 2000).

Clarifying the molecular mechanism of cell-cell adhesion will make it possible to clarify, for example, the mechanism of humectation and metastasis of carcinoma, and is expected to be applicable to diagnosis of carcinoma and also to development of medicines for carcinoma. For clarifying it, it is necessary to clarify all the details of the molecules that participate in cell-cell adhesion.

The invention of this application has been made in consideration of the current situation as above, and its one object is to provide a novel protein that participates in cell-cell adherens junctions.

Another object of the invention is to provide a gene-engineering material for producing the protein.

### DISCLOSURE OF THE INVENTION

To solve the problems noted above, this application provides a protein nectin-3 having the amino acid sequence of any of SEQ ID NO:2, 4 or 6.

The protein nectin-3 is derived from mice, including three splicing variants expressed by mouse genomic gene (these proteins are hereinafter referred to as nectin-3α, nectin-3β, nectin 3γ.

This application also provides a polynucleotide that encodes the protein nectin-3. The polynucleotide includes genomic DNA, mRNAs and cDNAs.

This application further provides cDNAs each encoding the three types of nectin-3, or that is, polynucleotides each having the base sequence of any of SEQ ID NO: 1, 3 or 5.

This application still further provides a recombinant vector having any of these polynucleotides.

This application still further provides an antibody against any of the three types of protein nectin-3.

Like nectin-1 and nectin-2, the protein nectin-3 of this invention is common to all mammals, existing in any and every mammal. Therefore, the protein nectin-3 and the polynucleotide encoding it are not limited to only mouse-derived ones.

### BRIEF DESCRIPTION OF THE DRAWINGS

FIG. 1 is to compare the amino acid sequences of nectin-3α, -3β and -3γ. The black background indicates the identical sequence among the three variants; the gray background indicates the identical sequence between nectin-3β and -3γ; the underline indicates the signal peptide; the double underlines indicate the transmembrane regions; and the asterisks indicate the glycosylated sites in asparagine.

FIG. 2 shows the experimental data of trans homo-interaction of nectin-3α. (A) indicates the expression level of nectin-3α, for which a cell lysate was subjected to development in SDS-PAGE followed by Western blot analysis with a polyclonal anti-nectin-3α antibody. (B) indicates cell aggregation activities of parental L cells (o) and nectin-3α-expressing L cells (●). (C) indicates cell aggregation activities of parental L cells (C1) and nectin-3α-expressing L cells (C2). The bar is 100 μm.

FIG. 3 shows the experimental data of cis homo-dimerization of nectin 3α. Nectin-3α-expressing cells were incubated in the presence or absence of BS3; each cell lysate was subjected to development in SDS-PAGE followed by Western blot analysis with a polyclonal anti-nectin-3α antibody. The arrow indicates the monomer; and the arrowhead indicates the dimer.

FIG. 4 shows the experimental data of mixed-cell aggregation activities of labeled nectin-1α-expressing L cells and unlabeled nectin-2α-expressing L cells (A1 to A3), labeled nectin-3α-expressing L cells and unlabeled nectin-1α-expressing L cells (B1 to B3), and labeled nectin-3α-expressing L cells and unlabeled nectin-2α-expressing L cells (C1 to C3). A1, B1 and C1 are the images in interference contrast microscopy; A2, B2 and C2 are the images in flourescence microscopy; A3, B3 and C3 are the data in statistical analysis. The bars are 40 μm.

FIG. 5 shows the images in immunofluorescence microscopy of co-cultured two L cell lines of nectin-1α-expressing L cells and -2α-expressing L cells (A1 to A3), nectin-3α-expressing L cells and -1α-expressing L cells (B1 to B3), and nectin-3α-expressing L cells and -2α-expressing L cells (C1 to C3). In A1 to B2, seen is nectin-1α; in A2 and C2, nectin-2α; in B1, nectin-3α (monoclonal antibody); in C1, nectin-3α (polyclonal antibody); and in A3, B3 and C3, merged. The bars are 10 μm.

FIG. 6 shows the experimental data of trans hetero-interaction affinity, for which a cell composition (labeled and unlabeled cells) of two-cell aggregates was quantitatively analyzed. A indicates the data of labeled nectin-1α-expressing L cells and unlabeled nectin-2α-expressing L cells; B indicates the data of labeled nectin-3α-expressing L cells and unlabeled nectin-1α-expressing L cells; and C indicates the data of labeled nectin-3α-expressing L cells and unlabeled nectin-2α-expressing L cells.

FIG. 7 shows the experimental data of cis hetero-dimerization nectin 3α with nectin-1α or -2α. A is for comparison of cis dimerization. A1 indicates the data of nectin-1α-expressing L cells (lane 1) and -1α/3α-expressing L cells (lane 2); A2 indicates the data of nectin-2α-expressing L cells (lane 1) and -2α/3α-expressing L cells (lane 2). B shows the data of immunoprecipitation. B1 indicates the data of nectin-1α/3α-expressing L cells; B2 indicates the data of nectin-2α/3α-expressing L cells. The cell extract is in lane 1; the supernatant is in lane 2; the precipitate is in lane 3. The arrows indicate monomers; and the arrowheads indicate the dimers.

FIG. 8 shows the experimental data of nectin-3 distribution in tissues. A shows the data of Northern blot analysis of nectin-1 (A1), nectin-2 (A2) and nectin-3 (A3). B shows the data of Northern blot analysis of nectin-3α (B1), nectin 3β (B2) and nectin-3γ (B3). Lane 1 is in heart; lane 2 is in brain; lane 3 is in spleen; lane 4 is in lung; lane 5 is in liver; lane 6 is in skeletal muscle; lane 7 is in kidney; and lane 8 is in testis.

FIG. 9 shows the experimental data of intracellular localization of nectin-3α in mouse small intestine absorptive epithelia. In A, seen is nectin-3α; in B, nectin-2; and in C, merged. The asterisks indicate the inner space of small intestines. The bar is 10 μm.

FIG. 10 shows the experimental data of direct binding of afadin to nectin-3α. The arrow indicates a fusion protein, GST-nectin-3α-CP; and the arrowhead indicates a fusion protein, MBP-afadin-PDZ.

### BEST MODE FOR CARRYING OUT THE INVENTION

A mouse protein, nectin-3α is encoded by 1650 bp cDNA having the base sequence of SEQ ID NO: 1, and this has the sequence of 549 amino acids as in SEQ ID NO: 2. The complete cDNA of the nectin-3α has the base sequence of 2178 bp as in SEQ ID NO: 7.

Nectin-3β is a protein encoded by 1533 bp cDNA having the base sequence of SEQ ID NO: 3, and this has the sequence of 510 amino acids as in SEQ ID NO: 4.

Nectin-3γ is a protein encoded by 1317 bp cDNA having the base sequence of SEQ ID NO: 5, and this has the sequence of 435 amino acids as in SEQ ID NO: 6.

These proteins nectin-3 can be obtained in any known method, for example, according to a method of isolating them from mouse and other mammal tissues; according to a method that comprises preparing peptides through chemical synthesis based on the amino acid sequences provided by this invention; or according to a method of recombinant DNA technology using the polynucleotides provided by this invention. Concretely, nectin-3 may be obtained through recombinant DNA technology as follows: An RNA is prepared through in-vitro transcription from a vector having a polynucleotide that encodes nectin-3, and the nectin-3 is expressed in vitro through in vitro translation using the RNA as a template. In the case of the polynucleotide encoding the nectin-3 being recombined with a suitable expression vector in a known manner, the nectin-3 encoded by the polynucleotide can be abundantly expressed in E. coli, B. subtilis, yeast, animal or plant cells, etc.

The protein nectin-3 of this invention can be expressed in microorganisms such as E. coli, in the following manner: The polynucleotide of this invention that encodes the protein is inserted into an expression vector having an origin replicavable in microorganisms, a promoter, a ribosome-binding site, a DNA cloning site and a terminator to construct a recombinant expression vector; then hose cells are transformed with the expression vector; and the resulting transformant cells are cultured. In that manner, the nectin-3 encoded by the polynucleotide can be abundantly produced in microorganisms. Alternatively, it may be expressed in the form of a fusion protein with any other protein segment. The resulting fusion protein is cleaved with a suitable protease, and only the protein segment encoded by the polynucleotide can be obtained.

The protein nectin-3 of this invention can be expressed in animal cells in the following manner: The polynucleotide of this invention that encodes the protein is recombined with an expression vector for animal cells having a promoter, a splicing region and a poly(A)-addition site, and the recombinant vector is introduced into animal cells, whereby the protein nectin-3 of this invention can be expressed in the thus-transformed animal cells.

The mouse protein nectin-3 obtained according to the method as above can be used, for example, as an antigen to form an antibody that specifically recognizes this protein.

The protein nectin-3 includes peptide fragments (of at lease 5 amino acid residues) having any partial amino acid sequence of the amino acid sequence of SEQ ID NO: 2, 4 or 6. These peptide fragments are also usable as an antigen for constructing the antibody.

The polynucleotide of this invention is a genomic gene of mammals that encodes any of the above-mentioned protein nectin-3α, β or γ. For example, it can be isolated from known genomic libraries, using the polynucleotide having the base sequence of any SEQ ID NO: 1, 3 or 5 or its partial sequence as a probe.

A polynucleotide of this invention may be cDNA characterized by having the base sequence of SEQ ID NO: 1, 3 or 5, and it encodes any of the above-mentioned nectin-3α, β or γ. Clones of the polynucleotides of this invention can be obtained with ease. Concretely, using an oligonucleotide probe synthesized on the bases of the base sequence of SEQ ID NO: 1, 3 or 5, mouse and other mammal cDNA libraries are screened. Alternatively, using the oligonucleotides as primers, the intended polynucleotides can be synthesized through polymerase chain reaction (PCR).

In general, mammal genes include many polymorphs owing to individual differences. Therefore, polynucleotides modified from those having a base sequence of any of SEQ ID NO: 1, 3 or 5 by adding, deleting and/or substituting one or more nucleotides therein with any nucleotide(s) are all within the scope of this invention. Similarly, proteins modified through the polynucleotide modification to add, delete and/or substitute one or more amino acid residues with any other amino acid residue(s) are also all within the scope of this invention, so far as they have the activity of the protein having the amino acid sequence of any of SEQ ID NO: 2, 4 or 6.

The polynucleotide of this invention includes DNA fragments (of at least 10 bp) having any partial base sequence of the base sequence of SEQ ID NO: 1, 3 or 5, and also polynucleotides comprising their antisense chains.

The antibody of this invention can be obtained as a polyclonal antibody or a monoclonal antibody in any known method of using the protein nectin-3 itself or its partial peptide as an antigen.

The following Examples are to show the experimental data to confirm the structure and the function of the nectin-3 of this invention.

### EXAMPLES

1. Procedures

1.1 Molecular Cloning of Mouse Nectin-3 cDNAs

From the EST database, three different types of mouse EST clones (AI1428160, AA492633, AA497887), which are similar to but are not the same as nectin-1 and -2, were amplified from a mouse brain cDNA (Clontech). Using the mixture of these cDNAs as a probe, a mouse cDNA library (Stratagene) was screened to obtain a full-length cDNA. For is DNA sequencing, used was a DNA sequencer (ABI 373).

1.2 Construction of Nectin-3 Expression Vectors

Vectors pCAGIPuro (J. Biol. Chem., 275:613-618, 2000), pCAGIPuro-FLAG, p-GEX-KG (Anal. Biochem., 192: 262-267, 1991), pMal-C2 (New England Biolabs Inc.) and pFastBAc1-Msp-Fc (J. Cell. biol., 145: 539-549, 1999) were used for constructing nectin-3 expression vectors mentioned below. The pCAGIPuro-FLAG was constructed by subcloning a prepro-trypsin signal peptide and the FLAG epitope of p FLAG-CMV1 (Eastman Kodak) into pCAGIPuro.

(a) pCAGIPuro-nectin-3α: 1-549 amino acids (full length) of SEQ ID NO: 2,

(b) pCAGIPuro-FLAG-nectin-3α: 56-549 amino acids of SEQ ID NO: 2,

(c) GST-nectin-3α-CP: 433-549 amino acids of SEQ ID NO: 2 (cytoplasmic region),

(d) GST-nectin-3α-CP-ΔC: 433-545 amino acids of SEQ ID NO: 2 (deletion of the C-terminal four amino acid residues),

(e) GST-nectin-3γˆ CP: 397-438 amino acids of SEQ ID NO: 6 (cytoplasmic region),

(f) pFastBac1-Msp-Fc-nectin-3α-EX: 56-400 amino acids of SEQ ID NO: 2 (extracellular region)

1.3 Construction of Transformant Cells

L cells (obtained from the Kyoto University) were cultured in a 10% fetal calf serum-containing DMEM medium to prepare parental L cells for transformation. Full-length human nectin-1α-expressing L cells (nectin-1α-L cells) and full-length mouse nectin-2α-expressing L cells (nectin-2α-L cells) were constructed according to the method described in references (J. Cell Biol., 145: 539-549, 1999; J. Biol. Chem., 275: 613-618, 2000). Full-length mouse nectin-3α-expressing L cells (nectin-3α-L cells) were constructed, using a recombinant vector pCAGIPuro-nectin-3α. Nectin-1α and FLAG-nectin-3α-coexpressing L cells (nectin-1α/3α-L cells), and nectin-2α and FLAG-nectin-3α-coexpressing L cells (nectin-2α/3α-L cells) were constructed by transfecting the nectin-1α-L cells and the nectin-2α-L cells, respectively, with pCAGIPuro-FLAG-nectin-3α, using a lipofectamine reagent (GIBCO BRL). Each cell line was cultured for 1 day, replated, and selected by culturing in the present of 5 μg/ml of puromycin (Sigma Chemical Co.).

1.4 Preparation of Antibodies

A rabbit antiserum (polyclonal antibody) against nectin-3α was raised against an antigen, GST-nectin-3α-CP. A rat monoclonal antibody against nectin-3 was raised against an antigen, fusion protein of the extracellular region of nectin-3α with IgG Fc. A rabbit polyclonal anti-nectin-1α antibody was prepared according to the method described in a reference (J. Cell Biol., 145: 539-549, 1999). Another rabbit polyclonal anti-nectin-1α antibody was raised against an antigen, synthetic peptide corresponding to the amino acid sequence 450-468 of nectin-1α, according to the method described in the reference (J. Cell Biol., 145: 539-549, 1999). A rat monoclonal anti-nectin-2 antibody was prepared according to the method described in references (J. Cell Biol., 145: 539-549, 1999; Exp. Cell Res., 235: 374-384, 1997); and mouse monoclonal and rabbit polyclonal anti-1-afadin antibodies were prepared according to the method described in references (J. Cell Biol., 139: 517-528, 1997; Oncogene 18: 1609-1618, 1999). A rat monoclonal anti-E-cadherin antibody was obtained from Dr. Takeichi (Kyoto University). A mouse monoclonal anti-FLAG antibody was bought from Eastman Kodak.

1.5 Other Procedures

Cell aggregation assay was done according to the method described in references (J. Cell Biol., 145: 539-549, 1999; J. Biol. Chem., 275; 613-618, 2000). For mixed-cell aggregation assay between two different L cell lines, one L cell line was prelabeled with DiI (Molecular Probe Inc., USA), as described (J. Cell Biol., 103: 171-187, 1986).

Chemical cross-linking was done according to the method described in references (Blood 92: 4602-4611, 1998; J. Biol. Chem., 275: 613-618, 2000). Immunoprecipitation was performed according to the method described in references (J. Cell Biol., 145: 539-549, 1999; J. Biol. Chem., 275: 613-618, 2000). Immunofluorescence microscopy of cultured cells was done according to the method described in references (J. Cell Biol., 139: 517-528, J. Cell Biol., 145: 539-549, 1999; J. Biol. Chem., 275: 613-618, 2000). Protein concentrations were determined with bovine serum albumin as a control, according to the method described in a reference (Anal. Biochem., 72: 248-254, 1976). SDS-PAGE was done as described (Nature, 227: 680-685, 1970).

Affinity chromatography was performed as follows: The MBP-fusion protein of the PDZ domain of afadin was immobilized on amylose resin beads (New England Biolabs Inc.) GST-nectin-3α-CP and GST-nectin-3α-CP-ΔC were separately applied to the affinity beads. After extensively washed with PBS (containing 0.1% Triton X-100), the beads were subjected to elution with PBS (containing 20 mM maltose, 0.1% Triton X-100).

2. Results

2.1 Cloning and Characterization of Nectin-3 cDNAs

The cDNA clone obtained from the mouse cDNA library has the base sequence of SEQ ID NO: 7, and encoded a protein (calculated molecular weight: 60,580) comprising 549 amino acids (SEQ ID NO: 2) in the 1647 bp coding regions (SEQ ID NO: 1). This clone contained all the above-mentioned EST clones. The protein was named nectin-3α.

The amino acid sequence of The nectin-3α had an N-terminal hydrophobic signal peptide (1-55 amino acids of SEQ ID NO: 2) and a transmembrane region (405-421 amino acids of SEQ ID NO: 2). The sites for N-linked glycosylation were detected at 73, 83, 125, 186, 222 and 331 amino acids. The nectin-3 contained three lg-like domains in the extracellular region and a C-terminal conserved motif in the cytoplasmic region (Table 1).

TABLE 1
C-Terminal Sequences of the Nectin Family

| Members | |
|---|---|
| Nectin-1α | SFISKKEWYV |
| Nectin-1β | VRTTEPRGEC |
| Nectin-2α | SLISRRAVYV |
| Nectin-2δ | DEFVSRAMYV |
| Nectin-3α | SVISRREWYV |
| Nectin-3β | LYINPREHYV |
| Nectin-3γ | LGQVRALEDT |

As in the above, these structural properties of nectin-3α are similar to those of nectin-1α, -1β, -2α and -2δ. The extent of homology varied with the regions of the molecules, but the amino acid sequence of the extracellular region of nectin-3α showed 35.9% and 30.7% identities to those of nectin-1 and nectin-2, respectively.

During the isolation of nectin-3α, two splicing variants (nectin-3β and -3γ) were isolated. The nectin-3β cDNA (coding region) has a base sequence of 1533 bps (SEQ ID NO: 3), and encodes a protein having an amino acid sequence of 510 amino acids (SEQ ID NO: 4, calculated molecular weight: 55,808). The extracellular region of nectin-3β (1-357 amino acids of SEQ ID NO: 4) is identical to that of nectin-3α, but its transmembrance and cytoplasmic regions (358-510 amino acids of SEQ ID NO: 4) are different from those of nectin-3α. However, nectin-3β also has a C-terminal conserved motif (Table 1).

The nectin-3γ cDNA (coding region) has a base sequence of 1317 bps (SEQ ID NO: 5), and encodes a protein having an amino acid sequence of 438 amino acids (SEQ ID NO: 6, calculated molecular weight: 47,259). The extracellular region, transmembrane region and cytoplasmic region of nectin-3γ are identical to those of nectin-3β, but nectin-3γ lacks the C-terminal conserved motif (Table 1).

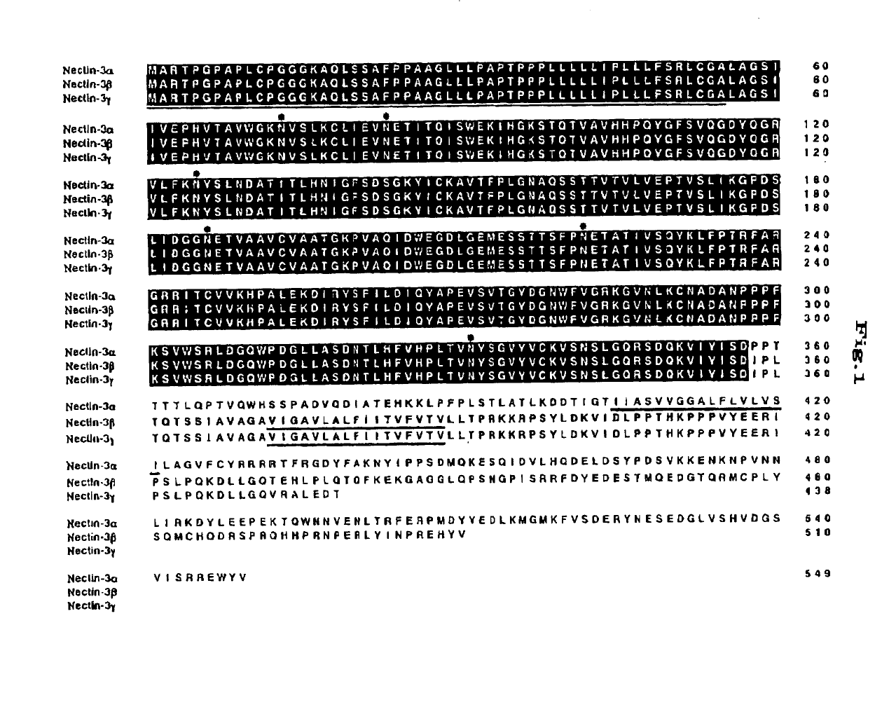

FIG. 1 is to compare the amino acid sequences (in one-letter coding) of these nectin-3α, -3β and -3γ.

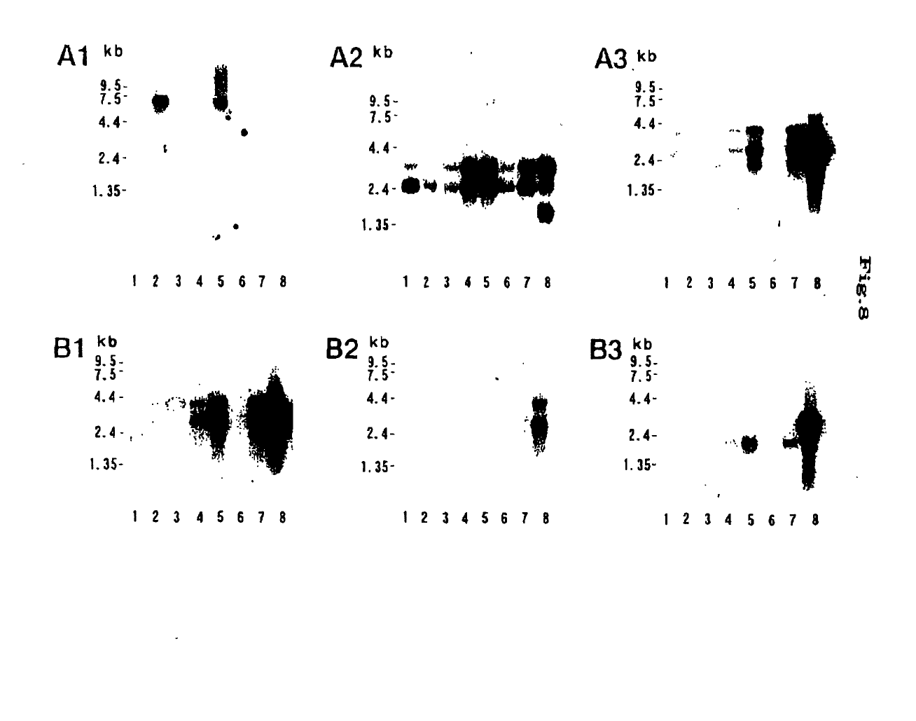

The following experiments are principally directed to nectin-3α. This is because the data in Northern blot analysis of various tissues confirm that nectin-3α is a major splicing variant (see FIG. 8, B1 to B3).

2.2 trans Homo-interaction and cis Homo-dimer Formation

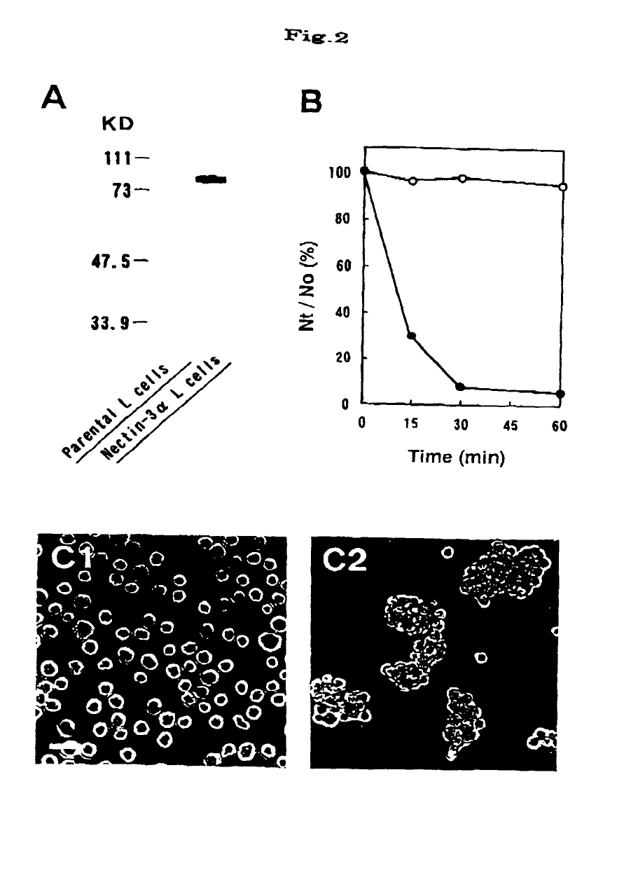

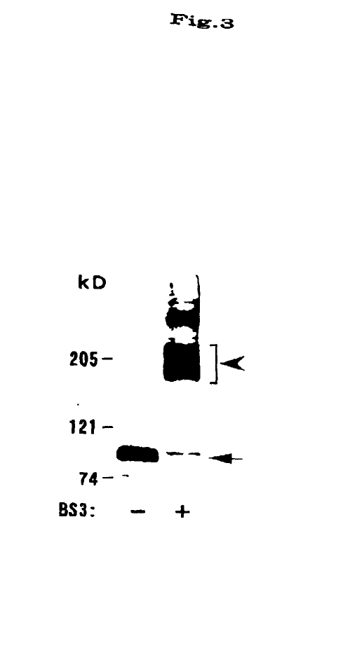

Studies with nectin-1α-L cells and nectin-2α-L cells confirm that nectin-1α and nectin-2α show cell-cell adhesion activity (trans homo-interaction) (J. Cell Biol., 145: 539-549, 1999; J. Biol. Chem., 275: 613-618, 2000). With that, here in examined was whether nectin-3α also shows the same activity. The polyclonal anti-nectin-3α antibody recognized two protein bands with molecular masses of about 100 kDA in the expression products by nectin-3α-L cells (FIG. 2A, FIG. 3). This may be dues to the different levels of the post-translational modification such as glycosylation. These molecular masses are different from the calculated molecular weight based on the deduced amino acid sequence. This difference may also be due to the glycosylation. The expression level of nectin-1α, -2α and -3α in each cell line was almost the same (data not shown).

Nectin-3α-L cells were tested for the cell aggregation activity of nectin-3α. As a result, the cell aggregation activity of nectin-3α varied time-dependently (FIG. 2B, C1, C2). EDTA added to nectin-3α did not have any influence on this activity of nectin-3α (data not shown). This confirms that the cell-cell adhesion activity of nectin-3α does not depend on Ca²⁺. These results indicate that nectin-3α is a Ca²⁺-independent homophilic CAM (cell adhesion molecule), like nectin-1α, -2α and -2δ (J. Cell Biol., 145: 539-549, 1999; Exp. Cell Res., 235: 374-384, 1997; Blood, 92: 4602-4611, 1998; J. Biol. Chem., 275: 613-618, 2000).

Nectin-1α and -2α are known to show cis homo-dimerization (J. Biol. Chem., 275: 613-618, 2000). With that, nectin-3α was also tested for the same activity. Nectin-3α-L cells were dissociated to a single-cell suspension and incubated with a cell surface cross-linker, BS3, and then subjected to Western blot analysis using a polyclonal anti-nectin-3α antibody. The cross-linking of the cell line resulted in the formation of additional bands with molecular masses of about 200 to 220 kDa that correspond to dimers (FIG. 3). Bands with higher molecular masses were also detected. Because the cross-linking was done in a single-cell suspension, it is most likely that the dimers and oligomers are derived from the cis homo-interaction rather that from the trans homo-interaction.

2.3 trans Hetero-interaction of Nectin-3α with Nectin-1α or -2α

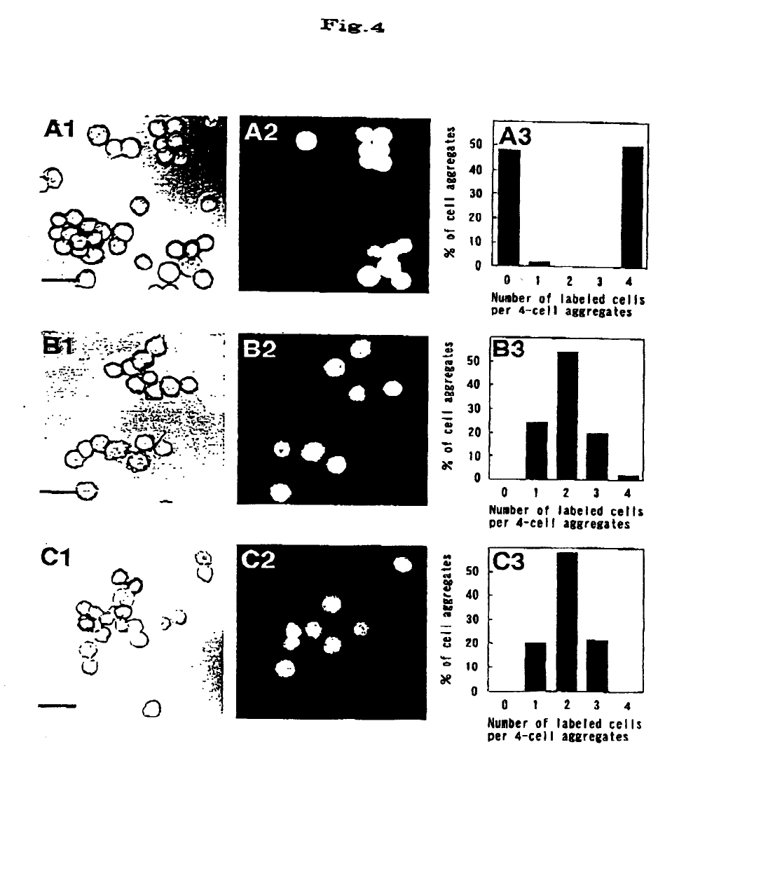

To examine whether each member of the nectin family shows heterophilic cell-cell adhesion activity (trans hetero-interaction), mixed-cell aggregation assay was performed. When DiI-labeled nectin-1α-L cells were mixed with unlabeled nectin-2α-L cells, the resulting aggregates exclusively consisted of the labeled cells alone or the unlabeled cells alone (FIG. 4 A1-A3). Few aggregates consisting of both the labeled and unlabeled cells were detected. In contrast, when DiI-labeled nectin-3α-L cells were mixed with unlabeled nectin-1α-L cells, the resulting aggregates consisted of both the labeled cells and the unlabeled cells (FIG. 4, B1-B3). This indicates that nectin-3α shows trans hetero-interaction with nectin-1α. The similar result was obtained with nectin-3α and nectin-2α (FIG. 4, C1-C3).

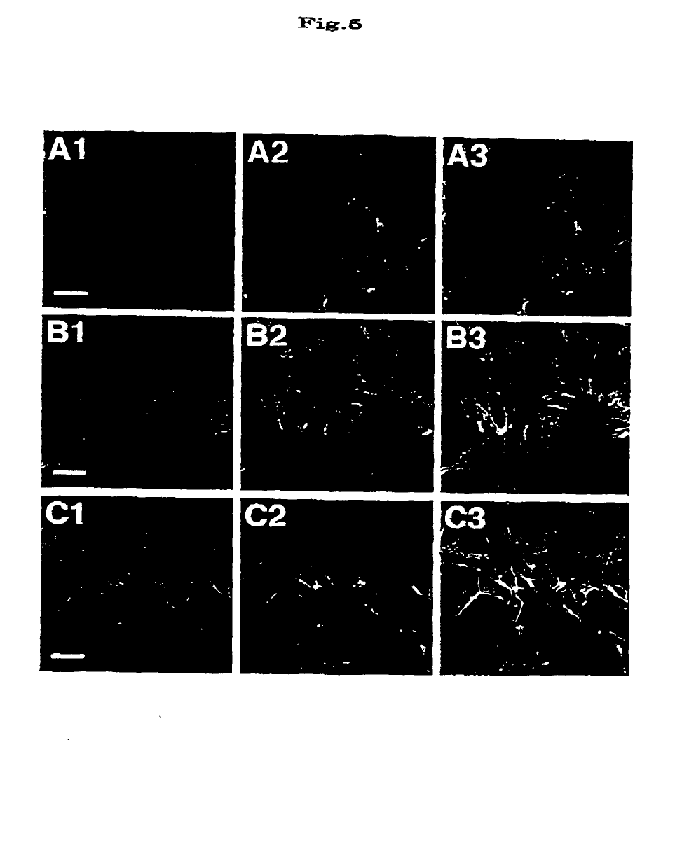

To confirm these results, immunofluorescence microscopy was performed. When nectin-1α-L cells were co-cultured with nectin-2α-L cells, nectin-1α and nectin-2α were localized in the cell-cell contact sites of the respective L cells (FIG. 5, A1-A3). However, neither nectin-1α or nectin-2α was detected in the contact sites of the two types of the L cells. In contrast, when nectin-3α-L cells were co-cultured with nectin-1α-L cells, nectin-3α and nectin-1α coexisted in the cell-cell contact site of the two types of the L cells (FIG. 5, B1-B3). The same result was obtained in co-culture of nectin-3α-L cells and nectin-2α-L cells (FIG. 5, C1-C3). These result indicate that nectin-3α shows trans hetero-interaction with nectin-1α and -2α, whereas nectin-1α does not trans hetero-interaction with nectin-2α.

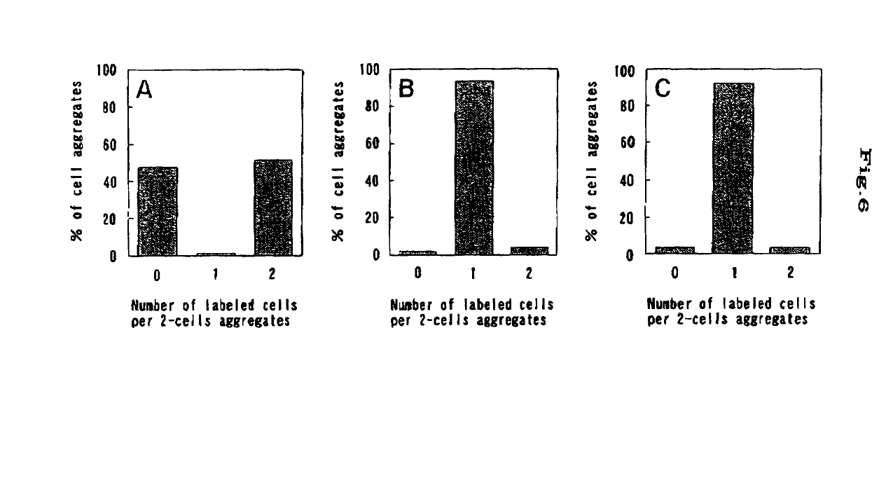

To determine whether each member of the nectin family prefers trans homo-interaction or trans hetero-interaction, two-cell aggregates were analyzed through mixed-cell aggregation assay. Consistent with the result in the analysis of two-cell aggregates, the mixture of nectin-1α-L cells and nectin-2α-L cells resulted in the formation of homotypic two-cell aggregates (FIG. 6A). In contrast, the mixture of nectin-1α-L cells and nectin-3α-L cells resulted in the formation of heterotypic two-cell aggregates, and few homotypic two-cell aggregates were found (FIG. 6B). The similar result was obtained from nectin-3α-L cells and nectin-2α-L cells were mixed (FIG. 6C). These results indicate that the affinity of trans hetero-interaction of nectin-3α with nectin-1α or -2α is obviously higher than that of trans homo-interaction of nectin-1α, -2α or -3α.

2.4 cis Hetero-dimerization of Nectin-3α with Nectin-1α or -2α

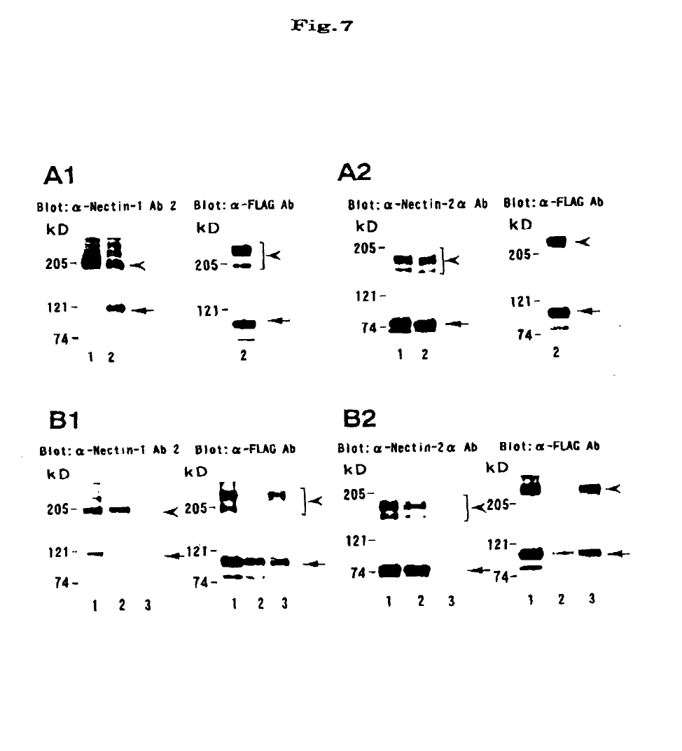

Next examined was whether nectin-3α forms a cis hetero-dimer with nectin-1α or -2α. Using nectin-1α/3α-L cells and nectin-2α/3α-L cells, FLAG-nectin-3α was expressed in the nectin-1α-L cells and the nectin-2α-L cells. As a result, the FLAG-nectin-3α expressed did not change the size of the cis dimers of nectin-1α or -2α (FIG. 7, A1 and A2). When nectin-1α/3α-L cells were subjected to cell surface cross-linking, followed by immunoprecipitation using the monoclonal anti-FLAG antibody, then nectin-1α was recovered in the supernatant and was not coimmunoprecipitated with FLAG-nectin-3α (FIG. 7, B1). the similar result was obtained with nectin-2α/3α-L cells (FIG. 7, B2). These results indicate that nectin-3α does not form a cis hetero-dimer with nectin-1α or -2α.

2.5 Tissue Distribution and Intracellular Localization of Nectin-3α

Consistent with previous reports (J. Virol., 66: 2807-2813, 1992; Gene 155: 261-265, 1995; Gene 159: 267-272, 1995), Northern blot analysis revealed that nectin-1 is abundantly expressed in brain whereas nectin-2 is ubiquitously expressed (FIG. 8, A1 and A2). Northern blot analysis using, as a probe, the coding region common to the three splicing variants of nectin-3 revealed some mRNA bands in various tissues (FIG. 8, A3). To determine the tissue distribution of each splicing variant, a cDNA probe specific to each variant was used. Nectin-3α gave about 5.2-kb, 3.8-kb, 3.3-kb and 2.7-kb mRNA bands, abundantly expressed in testis but slightly in other tissues (heart, brain, lung, liver and kidney) (FIG. 8, B1). Nectin-3β gave about 5.2-kb and 3.3-kb mRNA bands, expressed in testis (FIG. 8, B2). Nectin-3γ gave an about 3.3-kb mRNA band in testis, and an about 2.1-kb mRNA bank in lung, liver and kidney (FIG. 8, B3).

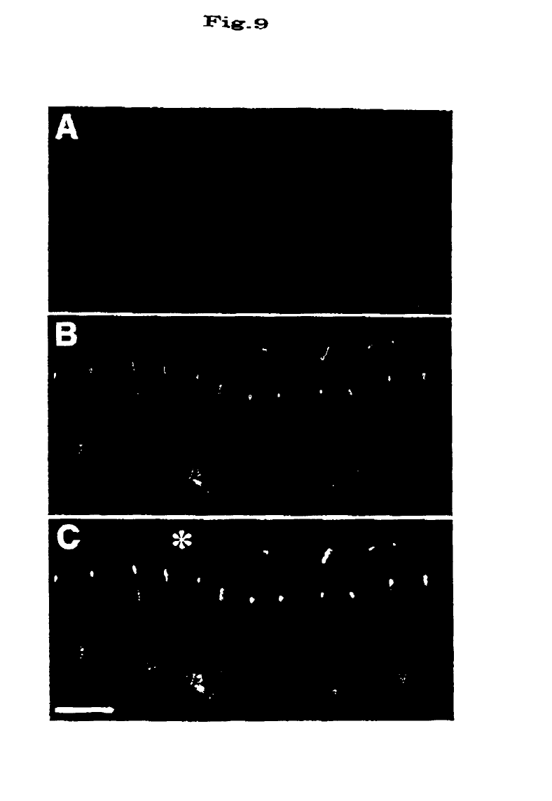

To determine the intracellular localization of nectin-3α, immunofluorescence microscopy was performed, using a polyclonal anti-nectin-3α antibody. Nectin-3α was colocalized with nectin-2 in the junctional complex regions in mouse small intestine absorptive epithelia (FIG. 9). These results suggest that nectin-3α is also localized at cadherin-based cell-cell AJs, like nectin-2 (J. Cell Biol., 145: 539-549, 1999).

2.6 Direct Binding of Nectin-3 to Afadin

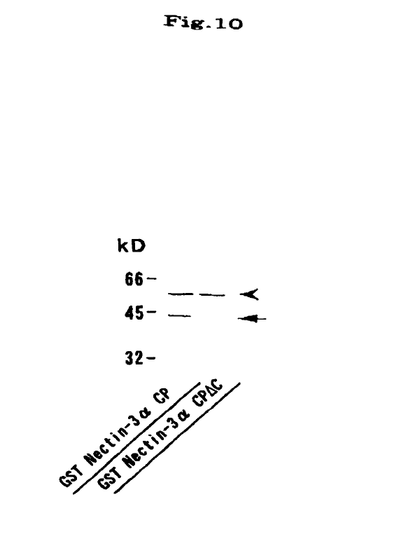

to confirm whether nectin-3α directly binds to afadin, affinity chromotography was performed. The GST-fusion protein in the cytoplasmic region of nectin-3α (GST-nectin-3α-CP) bound to the MBP-fusion protein in the PDZ domain of afadin )MBP-afadin-PDZ) immobilized on amylose resin beads (FIG. 10). The stoichiometry of binding of nectin-3α to afadin was about 1:1. In contrast, the GST-fusion protein in the C-terminal four amino acid residues-deleted cytoplasmic region of nectin-3α (GST-nectin-3α-CP-ΔC) did not bind to the affinity beads. Similarly, the GST-fusion protein in the cytoplasmic region of nectin-3γ that lacks the C-terminal conserved motif did not also bind to the affinity beads.

### INDUSTRIAL APPLICABILITY

As described in detail hereinabove, the invention of this application provides a novel protein nectin-3 that belongs to one and the same protein family to which nectin-1 and -2 belong. The protein provides important information for clarifying all aspects of the molecular mechanism in cell-cell binding systems, and, in addition, it leads to the possibility of clarifying the mechanism of, for example, humectation and metastasis of carcinoma, and is expected to be applicable to diagnosis of carcinoma for its malignancy and to a method for treating cases with carcinoma and also to development of medicines for carcinoma.

1. A protein nectin-3 having the amino acid sequence of SEQ ID NO: 2.

2. A protein nectin-3 having the amino acid sequence of SEQ ID NO: 4.

3. A protein nectin-3 having the amino acid sequence of SEQ ID NO: 6.

4. A polynucleotide encoding the protein nectin-3 of any of claims 1-3.

5. The polynucleotide as claimed in claim 2, which has the base sequence of SEQ ID NO: 1.

6. The polynucleotide as claimed in claim 2, which has the base sequence of SEQ ID NO: 3.

7. The polynucleotide as claimed in claim 2, which has the base sequence of SEQ ID NO: 5.

8. A recombinant vector having the polynucleotide of any of claims 4 to 7.

9. An antibody against the protein nectin-3 of any of claims 1 to 3.


SEQUENCE LISTING (14 sequences)

SEQ ID NO: 1 - DNA, 1650 bp, Mouse
  CDS (1)..(1650)
  ref <301>: AUTHORS: Satoh-Horikawa K. et al.
  ref <302>: TITLE: Nectin-3, a new member of immunoglobulin-like cell adhesion molecules that shows homophilic and heterophilic cell-cell adhesion activities.
  ref <303>: JOURNAL: J. Biol. Chem.
  ref <304>: VOLUME: 275
  ref <305>: ISSUE: 14
  ref <306>: PAGES: 10291-10299
  ref <307>: DATE: 2000-04-07
atggcgcggaccccgggcccggccccgttgtgtcctggaggcggcaaagcacaactttcctcggcgtttcctcccgcggccggactgctgctgccggccccgacgccgccgccgctgctgctgctgcttattcccctgcttctcttctcccggctctgtggtgccttagctggatcaattattgtggagccacatgtcacagcagtgtggggaaagaatgtttcattgaagtgtttaattgaagtgaatgaaactataacccagatctcatgggagaagatacatggcaaaagtacacagactgttgcagttcatcatcctcagtatggattctctgttcaaggagattatcagggaagagtcttgtttaaaaactattcacttaatgatgcaacaattactctgcataacataggcttctcagattctggaaaatatatatgcaaagccgttacattcccacttggaaatgctcagtcctctacaacagtgactgtgttagttgaacccacagtgagcctgataaaagggccggattctttaattgatggagggaatgagacagtagcagccgtttgtgtagcagccactggaaagccagtcgcacagattgactgggaaggtgatcttggtgaaatggaatctagtacaacttcttttcctaatgaaacagcaacgattgtcagccaatacaagctgtttcccacaagatttgctcgaggaaggcgaattacttgtgttgtaaaacatccagccttagaaaaggacattcgctactctttcatactagacatacagtatgctcctgaagtttcagtaacaggatatgatggaaattggttcgtgggaagaaaaggtgttaacctcaagtgtaatgctgatgcaaaccctccacccttcaagtccgtgtggagcaggttggatggacaatggcctgatggtttattggcgtcagataatactcttcattttgtccatccattgactgtcaattattctggcgtttatgtctgtaaagtatcaaattcccttggtcaaagaagtgatcaaaaggttatctacatttcagatcctcctaccaccaccacccttcagccgacagttcagtggcattcctcacctgctgacgtccaggatatagcaacagagcataaaaaattgccctttcctttgtcaactttggcaacacttaaggatgacacaattggcaccatcattgctagtgtagtgggtggggctctcttcttagtgcttgtgagcattttagctggggtattctgctataggagacgacggacgtttcgtggagactactttgccaaaaactacattccaccatcagacatgcagaaagaatcacagattgatgttcttcaccaggatgagctggattcttacccagacagtgtaaaaaaggaaaacaaaaatccagtaaacaacctgatccgcaaagactacttagaggagcctgagaaaactcagtggaataatgtagagaacctcactaggtttgaaagaccgatggattactatgaagatctaaaaatgggaatgaagtttgtcagtgatgaacgctacaatgaaagtgaagatggtttggtttctcatgtagatggctccgtaatttccaggagggagtggtatgtctaa

SEQ ID NO: 2 - protein, 549 aa, Mouse
MARTPGPAPLCPGGGKAQLSSAFPPAAGLLLPAPTPPPLLLLLIPLLLFSRLCGALAGSIIVEPHVTAVWGKNVSLKCLIEVNETITQISWEKIHGKSTQTVAVHHPQYGFSVQGDYQGRVLFKNYSLNDATITLHNIGFSDSGKYICKAVTFPLGNAQSSTTVTVLVEPTVSLIKGPDSLIDGGNETVAAVCVAATGKPVAQIDWEGDLGEMESSTTSFPNETATIVSQYKLFPTRFARGRRITCVVKHPALEKDIRYSFILDIQYAPEVSVTGYDGNWFVGRKGVNLKCNADANPPPFKSVWSRLDGQWPDGLLASDNTLHFVHPLTVNYSGVYVCKVSNSLGQRSDQKVIYISDPPTTTTLQPTVQWHSSPADVQDIATEHKKLPFPLSTLATLKDDTIGTIIASVVGGALFLVLVSILAGVFCYRRRRTFRGDYFAKNYIPPSDMQKESQIDVLHQDELDSYPDSVKKENKNPVNNLIRKDYLEEPEKTQWNNVENLTRFERPMDYYEDLKMGMKFVSDERYNESEDGLVSHVDGSVISRREWYV

SEQ ID NO: 3 - DNA, 1533 bp, Mouse
  CDS (1)..(1533)
  ref <301>: AUTHORS: Satoh-Horikawa K. et al.
  ref <302>: TITLE: Nectin-3, a new member of immunoglobulin-like cell adhesion molecules that shows homophilic and heterophilic cell-cell adhesion activities.
  ref <303>: JOURNAL: J. Biol. Chem.
  ref <304>: VOLUME: 275
  ref <305>: ISSUE: 14
  ref <306>: PAGES: 10291-10299
  ref <307>: DATE: 2000-04-07
  ref <308>: DATABASE ACCESSION NUMBER: GenBank accession No. AF195834
  ref <309>: DATABASE ENTRY DATE: 2000-04-13
atggcgcggaccccgggcccggccccgttgtgtcctggaggcggcaaagcacaactttcctcggcgtttcctcccgcggccggactgctgctgccggccccgacgccgccgccgctgctgctgctgcttattcccctgcttctcttctcccggctctgtggtgccttagctggatcaattattgtggagccacatgtcacagcagtgtggggaaagaatgtttcattgaagtgtttaattgaagtgaatgaaactataacccagatctcatgggagaagatacatggcaaaagtacacagactgttgcagttcatcatcctcagtatggattctctgttcaaggagattatcagggaagagtcttgtttaaaaactattcacttaatgatgcaacaattactctgcataacataggcttctcagattctggaaaatatatatgcaaagccgttacattcccacttggaaatgctcagtcctctacaacagtgactgtgttagttgaacccacagtgagcctgataaaagggccggattctttaattgatggagggaatgagacagtagcagccgtttgtgtagcagccactggaaagccagtcgcacagattgactgggaaggtgatcttggtgaaatggaatctagtacaacttcttttcctaatgaaacagcaacgattgtcagccaatacaagctgtttcccacaagatttgctcgaggaaggcgaattacttgtgttgtaaaacatccagccttagaaaaggacattcgctactctttcatactagacatacagtatgctcctgaagtttcagtaacaggatatgatggaaattggttcgtgggaagaaaaggtgttaacctcaagtgtaatgctgatgcaaaccctccacccttcaagtccgtgtggagcaggttggatggacaatggcctgatggtttattggcgtcagataatactcttcattttgtccatccattgactgtcaattattctggcgtttatgtctgtaaagtatcaaattcccttggtcaaagaagtgatcaaaaggttatctacatttcagacatcccgcttacgcagacctcatccatagcagtggctggagccgtgattggagctgtcctggccctcttcatcatcaccgtctttgtgactgtgttgctgacgcctcggaaaaagagaccgtcctatcttgacaaagtaatcgaccttccacctacacataagccaccccctgtatatgaagaacgaattccttctctccctcagaaagaccttcttggccagactgaacacttgcctttgcagactcagttcaaggagaaaggagctggtggtcttcagccctctaatggaccaattagcaggagatttgactatgaggatgagagcacaatgcaagaagatggaactcagcgcatgtgccccctttatagccagatgtgccaccaagaccgaagccctcgccaacatcacccacgcaaccccgagagactctacatcaacccacgagaacattatgtgtga

SEQ ID NO: 4 - protein, 510 aa, Mouse
MARTPGPAPLCPGGGKAQLSSAFPPAAGLLLPAPTPPPLLLLLIPLLLFSRLCGALAGSIIVEPHVTAVWGKNVSLKCLIEVNETITQISWEKIHGKSTQTVAVHHPQYGFSVQGDYQGRVLFKNYSLNDATITLHNIGFSDSGKYICKAVTFPLGNAQSSTTVTVLVEPTVSLIKGPDSLIDGGNETVAAVCVAATGKPVAQIDWEGDLGEMESSTTSFPNETATIVSQYKLFPTRFARGRRITCVVKHPALEKDIRYSFILDIQYAPEVSVTGYDGNWFVGRKGVNLKCNADANPPPFKSVWSRLDGQWPDGLLASDNTLHFVHPLTVNYSGVYVCKVSNSLGQRSDQKVIYISDIPLTQTSSIAVAGAVIGAVLALFIITVFVTVLLTPRKKRPSYLDKVIDLPPTHKPPPVYEERIPSLPQKDLLGQTEHLPLQTQFKEKGAGGLQPSNGPISRRFDYEDESTMQEDGTQRMCPLYSQMCHQDRSPRQHHPRNPERLYINPREHYV

SEQ ID NO: 5 - DNA, 1317 bp, Mouse
  CDS (1)..(1317)
  ref <301>: AUTHORS: Satoh-Horikawa K. et al.
  ref <302>: TITLE: Nectin-3, a new member of immunoglobulin-like cell adhesion molecules that shows homophilic and heterophilic cell-cell adhesion activities.
  ref <303>: JOURNAL: J. Biol. Chem.
  ref <304>: VOLUME: 275
  ref <305>: ISSUE: 14
  ref <306>: PAGES: 10291-10299
  ref <307>: DATE: 2000-04-07
  ref <308>: DATABASE ACCESSION NUMBER: GenBank accession No. AF195835
  ref <309>: DATABASE ENTRY DATE: 2000-04-13
atggcgcggaccccgggcccggccccgttgtgtcctggaggcggcaaagcacaactttcctcggcgtttcctcccgcggccggactgctgctgccggccccgacgccgccgccgctgctgctgctgcttattcccctgcttctcttctcccggctctgtggtgccttagctggatcaattattgtggagccacatgtcacagcagtgtggggaaagaatgtttcattgaagtgtttaattgaagtgaatgaaactataacccagatctcatgggagaagatacatggcaaaagtacacagactgttgcagttcatcatcctcagtatggattctctgttcaaggagattatcagggaagagtcttgtttaaaaactattcacttaatgatgcaacaattactctgcataacataggcttctcagattctggaaaatatatatgcaaagccgttacattcccacttggaaatgctcagtcctctacaacagtgactgtgttagttgaacccacagtgagcctgataaaagggccggattctttaattgatggagggaatgagacagtagcagccgtttgtgtagcagccactggaaagccagtcgcacagattgactgggaaggtgatcttggtgaaatggaatctagtacaacttcttttcctaatgaaacagcaacgattgtcagccaatacaagctgtttcccacaagatttgctcgaggaaggcgaattacttgtgttgtaaaacatccagccttagaaaaggacattcgctactctttcatactagacatacagtatgctcctgaagtttcagtaacaggatatgatggaaattggttcgtgggaagaaaaggtgttaacctcaagtgtaatgctgatgcaaaccctccacccttcaagtccgtgtggagcaggttggatggacaatggcctgatggtttattggcgtcagataatactcttcattttgtccatccattgactgtcaattattctggcgtttatgtctgtaaagtatcaaattcccttggtcaaagaagtgatcaaaaggttatctacatttcagacatcccgcttacgcagacctcatccatagcagtggctggagccgtgattggagctgtcctggccctcttcatcatcaccgtctttgtgactgtgttgctgacgcctcggaaaaagagaccgtcctatcttgacaaagtaatcgaccttccacctacacataagccaccccctgtatatgaagaacgaattccttctctccctcagaaagaccttcttggccaggtacgtgctctcgaagacacttaa

SEQ ID NO: 6 - protein, 438 aa, Mouse
MARTPGPAPLCPGGGKAQLSSAFPPAAGLLLPAPTPPPLLLLLIPLLLFSRLCGALAGSIIVEPHVTAVWGKNVSLKCLIEVNETITQISWEKIHGKSTQTVAVHHPQYGFSVQGDYQGRVLFKNYSLNDATITLHNIGFSDSGKYICKAVTFPLGNAQSSTTVTVLVEPTVSLIKGPDSLIDGGNETVAAVCVAATGKPVAQIDWEGDLGEMESSTTSFPNETATIVSQYKLFPTRFARGRRITCVVKHPALEKDIRYSFILDIQYAPEVSVTGYDGNWFVGRKGVNLKCNADANPPPFKSVWSRLDGQWPDGLLASDNTLHFVHPLTVNYSGVYVCKVSNSLGQRSDQKVIYISDIPLTQTSSIAVAGAVIGAVLALFIITVFVTVLLTPRKKRPSYLDKVIDLPPTHKPPPVYEERIPSLPQKDLLGQVRALEDT

SEQ ID NO: 7 - DNA, 2178 bp, Mouse
  CDS (197)..(1846)
  ref <301>: AUTHORS: Satoh-Horikawa K. et al.
  ref <302>: TITLE: Nectin-3, a new member of immunoglobulin-like cell adhesion molecules that shows homophilic and heterophilic cell-cell adhesion activities.
  ref <303>: JOURNAL: J. Biol. Chem.
  ref <304>: VOLUME: 275
  ref <305>: ISSUE: 14
  ref <306>: PAGES: 10291-10299
  ref <307>: DATE: 2000-04-07
  ref <308>: DATABASE ACCESSION NUMBER: GenBank accession No. AF195833
  ref <309>: DATABASE ENTRY DATE: 2000-04-13
gaattcggcacgagcgacggcggagtcgaggcagccgcgagcgctcggccgagtggcggcgggcggcgacggcgcaggagccgggggttgaggacacgcgcgctggcccttccgcgccgcggccgccgccgccgccgccacccagagcctgaggcgccggggcgcgggcgagcgggtgggccggggcaaggcgggcatggcgcggaccccgggcccggccccgttgtgtcctggaggcggcaaagcacaactttcctcggcgtttcctcccgcggccggactgctgctgccggccccgacgccgccgccgctgctgctgctgcttattcccctgcttctcttctcccggctctgtggtgccttagctggatcaattattgtggagccacatgtcacagcagtgtggggaaagaatgtttcattgaagtgtttaattgaagtgaatgaaactataacccagatctcatgggagaagatacatggcaaaagtacacagactgttgcagttcatcatcctcagtatggattctctgttcaaggagattatcagggaagagtcttgtttaaaaactattcacttaatgatgcaacaattactctgcataacataggcttctcagattctggaaaatatatatgcaaagccgttacattcccacttggaaatgctcagtcctctacaacagtgactgtgttagttgaacccacagtgagcctgataaaagggccggattctttaattgatggagggaatgagacagtagcagccgtttgtgtagcagccactggaaagccagtcgcacagattgactgggaaggtgatcttggtgaaatggaatctagtacaacttcttttcctaatgaaacagcaacgattgtcagccaatacaagctgtttcccacaagatttgctcgaggaaggcgaattacttgtgttgtaaaacatccagccttagaaaaggacattcgctactctttcatactagacatacagtatgctcctgaagtttcagtaacaggatatgatggaaattggttcgtgggaagaaaaggtgttaacctcaagtgtaatgctgatgcaaaccctccacccttcaagtccgtgtggagcaggttggatggacaatggcctgatggtttattggcgtcagataatactcttcattttgtccatccattgactgtcaattattctggcgtttatgtctgtaaagtatcaaattcccttggtcaaagaagtgatcaaaaggttatctacatttcagatcctcctaccaccaccacccttcagccgacagttcagtggcattcctcacctgctgacgtccaggatatagcaacagagcataaaaaattgccctttcctttgtcaactttggcaacacttaaggatgacacaattggcaccatcattgctagtgtagtgggtggggctctcttcttagtgcttgtgagcattttagctggggtattctgctataggagacgacggacgtttcgtggagactactttgccaaaaactacattccaccatcagacatgcagaaagaatcacagattgatgttcttcaccaggatgagctggattcttacccagacagtgtaaaaaaggaaaacaaaaatccagtaaacaacctgatccgcaaagactacttagaggagcctgagaaaactcagtggaataatgtagagaacctcactaggtttgaaagaccgatggattactatgaagatctaaaaatgggaatgaagtttgtcagtgatgaacgctacaatgaaagtgaagatggtttggtttctcatgtagatggctccgtaatttccaggagggagtggtatgtctaacagccactgacgcgacttcactatgtacaaggtttcattcacactagttgaccattttcagattgttcatactttttcttgaggaagaataagctttttcaagttgatttcgagcttactttttatattctgatctgacaaatgaaaatgtaaaacctgggttcaatgtatctgagctgctttacagttttcactgctatactactgtctcaagatttaaattctaatgcagagtactttattggtctgaggcacacaggtaagaaagatgtcaacgttaaatgtatgacgtttttggtacaaaaattaaaaaaaaaaaaaaaaaactcgag

SEQ ID NO: 8 - protein, 10 aa, Artificial Sequence
  Description of Artificial Sequence: C-Terminal Sequence of Nectin-1alpha
SFISKKEWYV

SEQ ID NO: 9 - protein, 10 aa, Artificial Sequence
  Description of Artificial Sequence: C-Terminal Sequence of Nectin-1beta
VRTTEPRGEC

SEQ ID NO: 10 - protein, 10 aa, Artificial Sequence
  Description of Artificial Sequence: C-Terminal Sequence of Nectin-2alpha
SLISRRAVYV

SEQ ID NO: 11 - protein, 10 aa, Artificial Sequence
  Description of Artificial Sequence: C-Terminal Sequence of Nectin-2delta
DEFVSRAMYV

SEQ ID NO: 12 - protein, 10 aa, Artificial Sequence
  Description of Artificial Sequence: C-Terminal Sequence of Nectin-3alpha
SVISRREWYV

SEQ ID NO: 13 - protein, 10 aa, Artificial Sequence
  Description of Artificial Sequence: C-Terminal Sequence of Nectin-3beta
LYINPREHYV

SEQ ID NO: 14 - protein, 10 aa, Artificial Sequence
  Description of Artificial Sequence: C-Terminal Sequence of Nectin-2gamma
LGQVRALEDT